(19) United States
(12) Patent Application Publication
(10) Pub. No.: US 2022/0323657 A1
(43) Pub. Date: Oct. 13, 2022

(54) Pumpless Silent Negative Pressure System

(71) Applicant: Freemom Technology Co., Ltd., Taipei City (TW)

(72) Inventor: Shyh-Jer Huang, Tainan City (TW)

(21) Appl. No.: 17/709,587

(22) Filed: Mar. 31, 2022

(30) Foreign Application Priority Data
Apr. 9, 2021 (TW) 110112997

(51) Int. Cl. A61M 1/06

(52) U.S. Cl. CPC A61M 1/0697; A61M 1/064

(57) ABSTRACT

A pumpless silent negative pressure system comprises: a horn cover, a cylinder is provided to its lower end; a software arranged inside the horn cover, the rear end of the software is provided with one or more coils. The software's center is provided with a cavity for generating negative pressure, the front end of the software is provided with an opening for adhering to the object to be sucked, the rear end of the software is extended with a cavity inside the cavity and having a return stress rebound and a single or a single or a plurality of elastic inner concave parts of a plurality of negative pressure chambers, the front end surface of the elastic inner concave part forms a negative pressure chamber film and a stroking type promoting milk film, and the end of the elastic inner concave part is provided with a magnet.

### FIELD

The present invention relates to a silent negative pressure system and, in particular, to a pumpless silent negative pressure system.

### BACKGROUND

Pressing a breast pump is in order to generate negative pressure to suck the milk. This negative pressure is usually achieved by a pumping motor. However, the sound of the conventional pumping motor cannot be completely silent, which makes the mothers psychologically burdened when using the breast pump. In addition, it is easy to wake up the sleeping father or baby when expressing milk in the night. Even if the current market milking machine emphasizes low noise, it still has a volume of at least about 40 dB, which cannot be completely silent. Young children are very sensitive to the sound of the motor, so parents often have to struggle with their children when using the current nasal aspirator, and both sides are struggling. Traditional breast pump includes a main unit, a squeezing unit, a milk collection bag, a stop valve unit and a drive unit. The air is pumped through the motor in the main unit, so that the negative pressure chamber of the software generates negative pressure and then compresses and deforms, which drives the milk collecting chamber to generate negative pressure to suck out the milk. However, since the motor drive will generate noise, special sound insulation treatment is required to reduce the trouble for users as mentioned above.

For example, China patent number: CN103182103B, titled “Electric Breast Pump”, according to its abstract content, it is mainly as follows: the invention includes the body, the hood, the negative pressure generating mechanism, the negative pressure regulating mechanism and the pressure display mechanism, and the breast pump connected with the negative pressure generating mechanism through the hose and the milk collecting mechanism; negative pressure generating mechanism includes stepper motor and cylinder; negative pressure regulating mechanism includes negative pressure regulating mechanism and the rubber ring, the commander and the receiver, the negative pressure adjusting rubber ring can be rotated radially on the cylinder, and the outer end of the commander is fixed on the negative pressure adjusting rubber ring to drive the negative pressure adjusting rubber ring, the receiver is nested in the commander and between the cylinder and fixedly connected with the cylinder. By selecting a stepper motor and setting a negative pressure adjustment mechanism, stable negative pressure can be generated, and the piston frequency can be controlled by the stepper motor, which is closer to the baby's sucking frequency and improves the comfort during breastfeeding; the pressure display mechanism can make users to adjust the size of negative pressure more intuitively. In addition, according to the content defined in claim 1 of its claim, it is that an electric breast pump, comprises body, hood, negative pressure generating mechanism, and by Xi Rujiru mechanism that flexible pipe is connected with negative pressure generating mechanism; it is characterized in that: Also comprise negative pressure regulating mechanism and pressure display organ; described negative pressure generating mechanism comprises motor, piston, cylinder and cylinder fixture; motor is arranged in body, and cylinder fixture is movably arranged on body, and it is outer and be connected between motor and cylinder fixture that piston and cylinder are arranged on body; the inner of piston is connected with motor transmission by crank, and outer end activity is nested in cylinder; cylinder is axially through tubular structure part, and outer end and the cylinder fixture of cylinder connect, and cylinder is provided with leakage hole; cylinder fixture is the bent structural member of hollow, and the outer end of cylinder fixture collects newborn mechanism connection by flexible pipe and suction breast; described negative pressure regulating mechanism comprises negative pressure and regulates elastic tape, instruction device and receptor, negative pressure regulates elastic tape movable set can radial rotating on cylinder, the outer end of instruction device is fixedly set in negative pressure and regulates on elastic tape negative pressure can be driven to regulate elastic tape to rotate, and receptor is nested in the inner of instruction device and is fixedly connected with cylinder with between cylinder; be inlaid with magnet steel in instruction device, receptor is inlaid with Hall element, magnet steel is corresponding with Hall element to be arranged; described pressure display organ is LCDs, and this LCDs is arranged on body surface and is connected with the Hall element signal of telecommunication; described instruction device is cylindrical, and internal diameter is three sections of stepped ramp types, and the outer section that internal diameter is less regulates elastic tape to mate with negative pressure, and the side of stage casing inwall evenly has 5 sulculuses, is placed with magnet steel respectively in groove; The stage casing of described receptor and instruction device is nested to be connected, the outer wall of receptor side has 5 sulculuses, Hall element is placed with respectively in groove, each Hall element is supporting with each magnet steel respectively correspondingly to be arranged and telecom-connects with LCDs respectively, and described negative pressure regulates that the outer section of elastic tape and instruction device is nested to be connected; described cylinder is being provided with one section of radial annular groove with the position that negative pressure regulates elastic tape to be connected, and the inner chamber that one end of annular groove is provided with at least one leakage hole and cylinder is through; described negative pressure regulates the inwall of elastic tape to be provided with an axial pass through, and this axial pass trough can be communicated with the radial annular groove on cylinder. It is to expose an electric breast pump by exposing the habit in the front, inlay the magnetic steel in the commander, and inlay the Hall element in the receiver, and use the negative pressure rubber ring to rotate to adjust the negative pressure. The components are many and complicated, the mute cannot be achieved.

For example, China patent number: CN109125822A, the main content of the invention patent for “a negative pressure relief device on a breast pump” is as follows: the present invention provides the negative pressure relievers on a kind of breast pump, belong to mother and baby's technical field of tools. Breast pump includes the hose two of sucker and connecting sucker, this reliever includes the pump housing and motor, a runner is connected with by spline slide on the output shaft of motor, a working chamber is offered on the pump housing, runner rotates in working chamber, there is the suction hole of connection hose two and working chamber on the pump housing, suction hole has the insertion section of a taper close to the opening of working chamber one end, several permanent magnets being located on the outside of insertion section are provided on the inner wall of working chamber, the iron block of several and permanent magnets face is provided on runner, the plug that can be plugged in insertion section is further fixedly arranged on runner. Also as defined in claims 1-4 of its request: the negative pressure reliever on a kind of breast pump, which is characterized in that breast pump includes sucker (1) and connecting sucker (1) Hose two (22), this reliever include the pump housing (6) and motor (71), pass through spline slide on the output shaft of the motor (71) It is connected with a runner (72), is offered a working chamber (73) on the pump housing (6), runner (72) rotation is in working chamber (73) It is interior, there is the suction hole (74) of connection hose two (22) and working chamber (73), the suction hole (74) is close on the pump housing (6) The opening of working chamber (73) one end has the insertion section (75) of a taper, is provided on the inner wall of the working chamber (73) several A permanent magnets (76) on the outside of insertion section (75) are provided with several and permanent magnets (76) face on the runner (72) Iron block (77) is further fixedly arranged on the plug (78) that can be plugged in insertion section (75) on the runner (72). The negative pressure reliever on a kind of breast pump according to claim 1, which is characterized in that the motor (71) it is defeated It is connected between shaft and runner (72) preloading spring (79), the preloading spring (79), which has, drives plug (78) far from slotting Enter the restoring force of portion (75): When motor (71) stop transport, the pre-tightening force of the preloading spring (79) is less than permanent magnets (76) to iron The adsorption capacity of block (77). The negative pressure reliever on a kind of breast pump according to claim 1 or claim 2, which is characterized in that the pump housing (6) and Motor (71) is each attached in a box body (8), and several vent holes communicated with working chamber (73) are offered on the box body (8) (81). The negative pressure reliever on a kind of breast pump according to claim 3, which is characterized in that in the box body (8) also is provided with battery, the battery is electrically connected with motor (71). It is a kind of external pump type milk collecting device that is disclosed by the front of the habit. It uses the runner to embed the permanent magnet block and the iron block to attract to ease the negative pressure and reduce the discomfort of the user. The main reason is to avoid the pressure too much. A kind of softener derived from the fast, its components are many and complicated, and it cannot achieve the effect of silence.

For example, according to the invention patent of the China patent number CN107980642A “Electronic Negative Pressure Pulsator for Milking”, according to the scope of claim 1-3, it is described as follows: A kind of 1. electronics negative pressure pulsator of milking, it is characterized in that: Including lid (1), electromagnetic valve coil (2) two, electricity Magnet valve core (3), electromagnetism valve seals (4), electric crossover board (5), solenoid valve pedestal (6), sealing rubber pad (7), floating core cavity body (8), vacuum outlet main body (10), floating core (11), sealing rubber ring (12), sealing diaphragm (13), negative pressure main channel (14) and close Packing (16), the lid (1) are equipped with external pressure hole (00), the lid (1), solenoid valve pedestal (6), sealing rubber pad (7), core cavity body (8), vacuum outlet main body (10) and negative pressure main channel (14) are floated to be sequentially connected. The electromagnetic valve coil (2) is electrically connected with electric crossover board (5), and solenoid valve conduction hole is equipped with the top of the solenoid valve (2) (21), the electromagnetic valve core (3) is located in electromagnetic valve coil (2), and the electromagnetic valve coil (2) passes through electromagnetism valve seals (4) on solenoid valve pedestal (6), the electric crossover board (5) is also mounted on solenoid valve pedestal (6); Solenoid valve (2) installation site on the solenoid valve pedestal (6) is equipped with cavity one (61) and cavity two (62), the type chamber one (61) is equipped with negative pressure hole one (611) and deflector hole one (612), and the cavity two (62) is equipped with negative pressure hole two (621) With deflector hole two (622); Electromagnetic valve core (3) difference face negative pressure hole one (611) and negative pressure hole two (621) in described two electromagnetic valve coils (2); Be respectively equipped with the sealing rubber pad (7) with negative pressure hole one (611), deflector hole one (612), negative pressure hole two (621) and Deflector hole three (71), negative pressure hole three (72), negative pressure hole four (73) and the deflector hole six (74) of deflector hole two (622) connection; The floating core cavity body (8) is equipped with cavity three (81), cavity four (82), through hole one (83), through hole two (84), seal groove (85) and center square hole one (86), the cavity three (81) are equipped with through hole one (83), are set on the cavity four (82) There is through hole two (84), the through hole one (83) and through hole two (84) are connected with deflector hole three (71) and deflector hole six (74) respectively, The seal groove (85) is located at the outside of center square hole one (86) and is connected with negative pressure hole three (72) and negative pressure hole four (73); The vacuum outlet main body (10) include exterior communicating pipe one (101), exterior communicating pipe two (102), stomata one (103) and Stomata two (104), the stomata one (103) connect with exterior communicating pipe one (101), and the stomata two (104) connects with outside Pipe two (102) connects, and described one (103) place of stomata and stomata two (104) are equipped with diaphragm groove one (105) and diaphragm groove two (106), the sealing diaphragm (13) includes sealing diaphragm one (131), sealing diaphragm two (132) and centre bore two (133), described sealing diaphragm one (131) is located in diaphragm groove one (105), and the sealing diaphragm two (132) is located in diaphragm groove two (106), institute State and negative pressure square hole (107) is additionally provided with vacuum outlet main body (10), the negative pressure square hole (107) is located at vacuum outlet main body (10) Center, the diaphragm groove one (105) is equipped with connectivity slot one (1051), and the diaphragm groove two (106) is equipped with connection Groove two (1061), the connectivity slot one (1051) and connectivity slot two (1061) are connected with negative pressure square hole (107); The floating core (11) includes floating core one (111) and floating core two (112), the floating core one (111) and floating core two (112) bottom Sealing rubber ring (12) is mounted on, the floating core one (111) is connected through stomata one (103) with sealing diaphragm one (131); Institute Floating core two (112) is stated to be connected with sealing diaphragm two (132) through stomata two (104): The negative pressure main channel (14) is installed in vacuum outlet main body (10) by gasket (16), the negative pressure main channel (14) negative tube (141) is equipped with, the negative tube (141) is located at the center of negative pressure main channel (14); The negative pressure main channel (14) is equipped with negative pressure trough (142), and the negative pressure trough (142) is connected with negative tube (141) and position In negative pressure main channel (14) center. The electronics negative pressure pulsator of milking according to claim 1, it is characterized in that: Dustproof cover (15) is further included, the dustproof cover (15) is installed on lid (1), and the dustproof cover (15) is equipped with dustproof cover air inlet pipe (151). The electronics negative pressure pulsator of milking according to claim 1, it is characterized in that: Further include sponge buckle closure (17) With dustproof sponge (18), the dustproof sponge (18) is installed on sponge buckle closure (17), and the sponge buckle closure (17) is located at dust-proof cover between (15) and lid (1). It was revealed by the former that the habit was used as an electronic negative pressure pulsator for milking, and the structure was complicated and could not achieve a mute effect.

It is a conventional product that has been disclosed before. Since there is still room for improvement in terms of quietness and simplification of components, this is where the industry and consumers are eager to make breakthroughs.

### SUMMARY OF THE DISCLOSURE

In order to solve the above-mentioned deficiencies of the prior art, the main purpose of the present invention is to provide a silent negative pressure system that does not require a pump to drive. On the surface, a negative pressure cavity film and a stroking pro-emulsifying film are formed, and the back end of the software is provided with a coil that interacts with a magnet, in order to overcome the difficulties in the prior art.

The secondary purpose of the present invention is to provide a silent negative pressure system that does not require a pump to drive, utilizes a current to drive the coil to generate a magnetic field, and provides the elastic inner recess to deform and the cavity to generate a silent negative pressure.

Another object of the present invention is to provide a silent negative pressure system that does not require a pump to drive, effectively improve its ease of use and mute effect, and save the conventional motor and pressure relief valve.

The problem to be solved by the present invention is, for example, the Republic of China Invention Patent No. 1675675 (Japanese Patent No. 6691591), which discloses an invisible milking device. However, since the motor drive will generate noise, special sound insulation treatment is required to reduce the user Troubled; such as the Chinese Mainland Announcement: CN103182103B “Electric Breast Pump” Invention Patent, Chinese Mainland Invention Patent Publication No.: CN109125822A “A Negative Pressure Relief Device on a Breast Pump” Invention Patent, Chinese Mainland Publication No. CN107980642A “Milk Milking”. The electronic negative pressure pulsator used in the invention patent, its components are many and complex, and it cannot achieve mute.

The technical means of solving the problem, in order to achieve the above-mentioned purpose, the present invention provides a kind of silent negative pressure system that does not need to be driven by a pump, it is characterized in that, comprises:
a horn cover, wherein the lower end of which is provided with a cylinder; a software arranged inside the horn cover, wherein the rear end of the software is provided with one or more coils,
wherein the center of the software is provided with a cavity for generating negative pressure, the front end of the software is provided with an opening for adhering to the object to be sucked, the rear end of the software is extended with a cavity inside the cavity and having a return stress rebound and a single or a single or a plurality of elastic inner concave parts of a plurality of negative pressure chambers, the front end surface of the elastic inner concave part forms a negative pressure chamber film and a stroking type promoting milk film, and the end of the elastic inner concave part is provided with a magnet corresponding to the coil;
wherein by using current to drive the coil to generate a magnetic field, interaction with the magnet is provided, the elastic inner recess is deformed and a silent negative pressure in the cavity is generated.

Preferably, the inner section of the lower edge of the cavity of the software is provided with a flow channel that communicates with the cavity, and the cylinder of the horn cover is provided with a collection groove and communicated with the flow channel.

Preferably, the middle section of the lower edge of the cavity of the software is provided with a side flow channel communicating with the cavity, and a blocking device is protruded from the upper end of the side flow channel.

Preferably, the lower end of the side flow channel of the cavity meets the lower end of the flow channel and communicates with the collection tank and is provided with a check valve.

Preferably, the coil of the software controls the magnetic force of the coil through the magnitude of the current.

Preferably, the collecting tank of the cylinder is provided with a dairy collecting product.

Preferably, a sealing clip is arranged at the lower end of the collecting tank of the cylinder.

Preferably, the milk collection system is a milk collection bag or a feeding bottle.

Preferably, this dairy collecting bag of described dairy collecting product is provided with an opening at its one end, and this opening is provided with a sealing clip.

In contrast to the effect of the prior art, the present invention utilizes the back end of the software to extend the elastic inner concave portion with the magnet at the inner end, and the front end surface of the elastic inner concave portion forms the negative pressure chamber film and the touch-type emulsification-promoting film. The back end of the software is provided with the coil that interacts with the magnet; further, it is achieved that the coil is driven by current to generate a magnetic field, the elastic inner recess is deformed, and the cavity generates a mute negative pressure; the convenience of use and mute effect are effectively improved, and the conventional motor and pressure relief valve will greatly expand the utilization of the industry and will be novel and progressive.

### BRIEF DESCRIPTION OF THE DRAWINGS

FIG. 1: is an exploded perspective view of the present invention.

The second figure: a three-dimensional combined view of the present invention.

FIG. 3: It is a combined cross-sectional view of the present invention.

The fourth figure: an embodiment of the present invention and the object to be sucked (breast, nipple).

FIG. 5: It is the top view of the host of the present invention.

The sixth figure: the bottom view of the host of the present invention.

FIG. 7: It is an embodiment of the invention that the number collecting groove of the cylinder is provided with a sealing clip.

### DETAILED DESCRIPTION

The present invention is hereby described in detail as follows in the form of an embodiment in conjunction with the accompanying drawings, and the drawings used in the text are only for the purpose of illustrating and assisting the description, and are not necessarily the real scale and accuracy after the implementation of the present invention Therefore, the scope of the invention should not be limited to the actual implementation of the patented scope of the present invention based on the proportions and configuration relationships of the attached drawings.

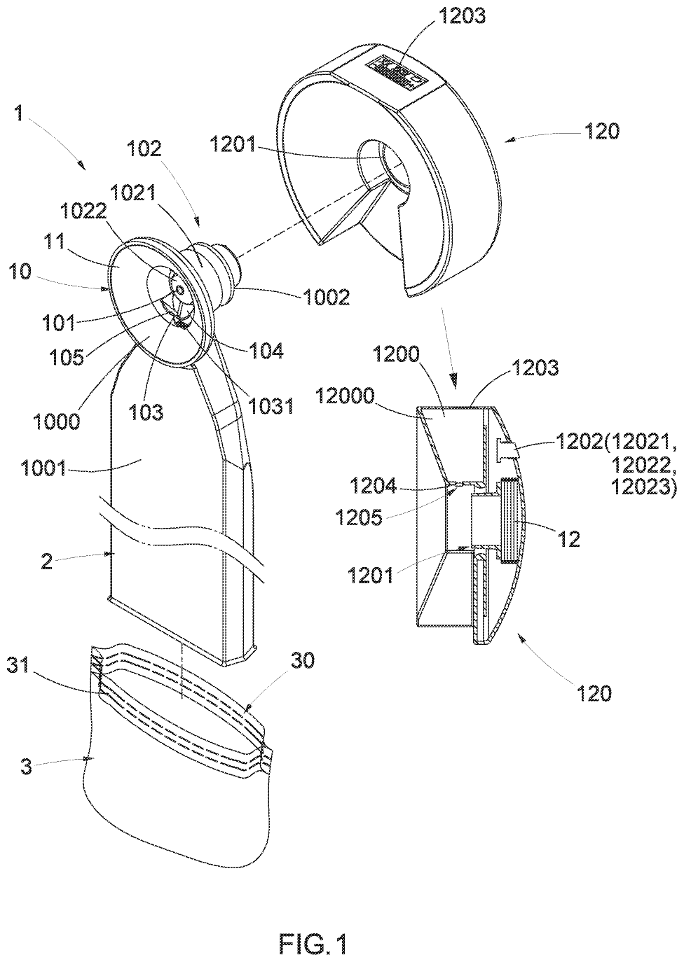

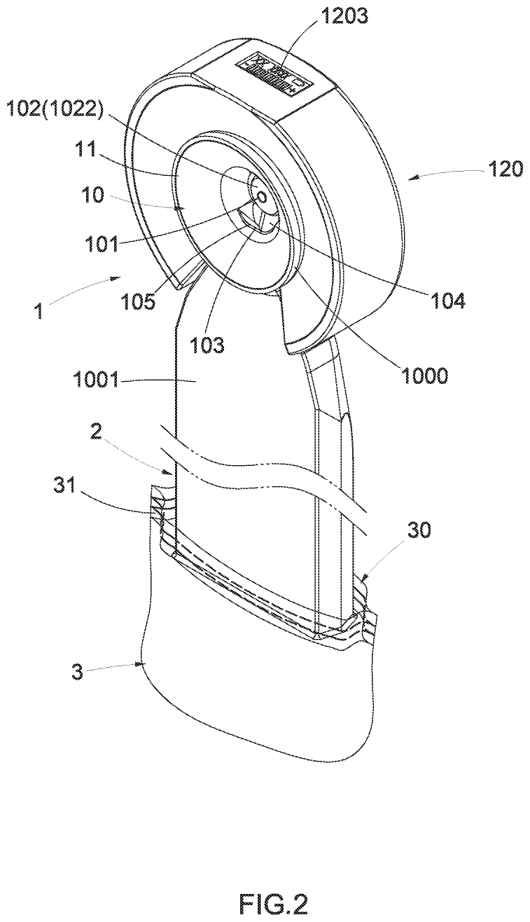

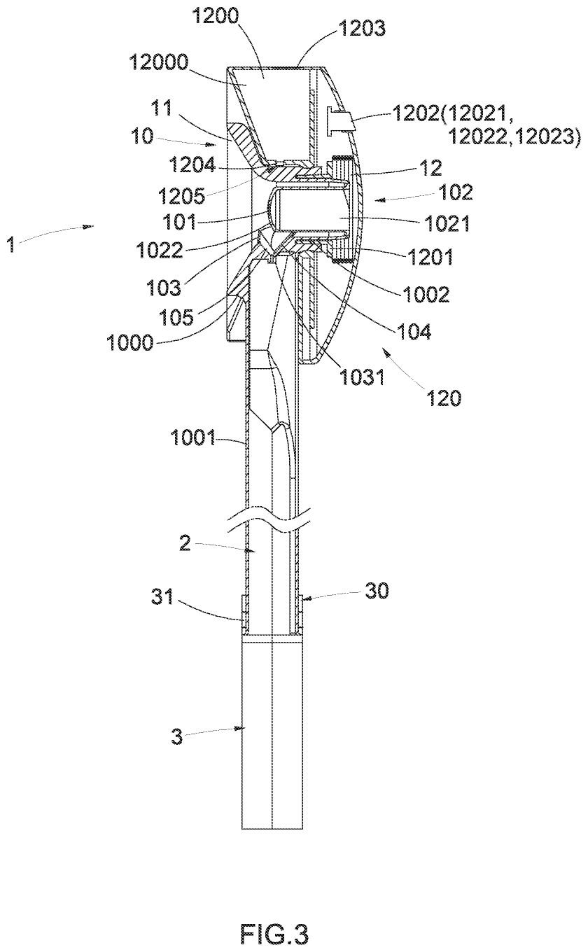

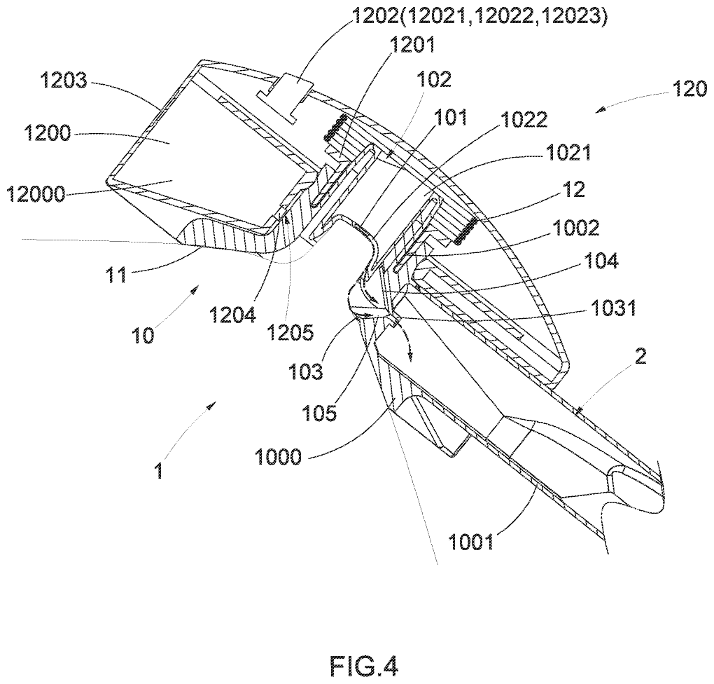

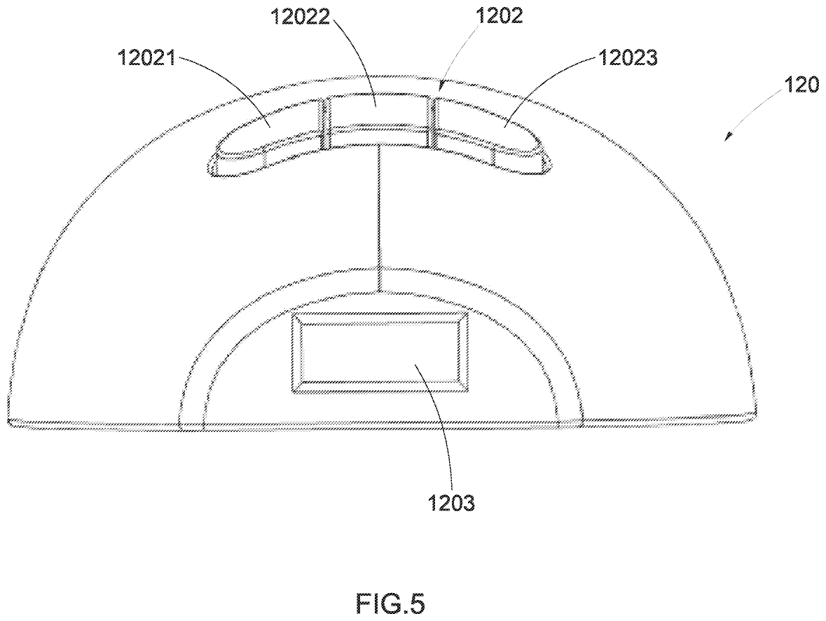

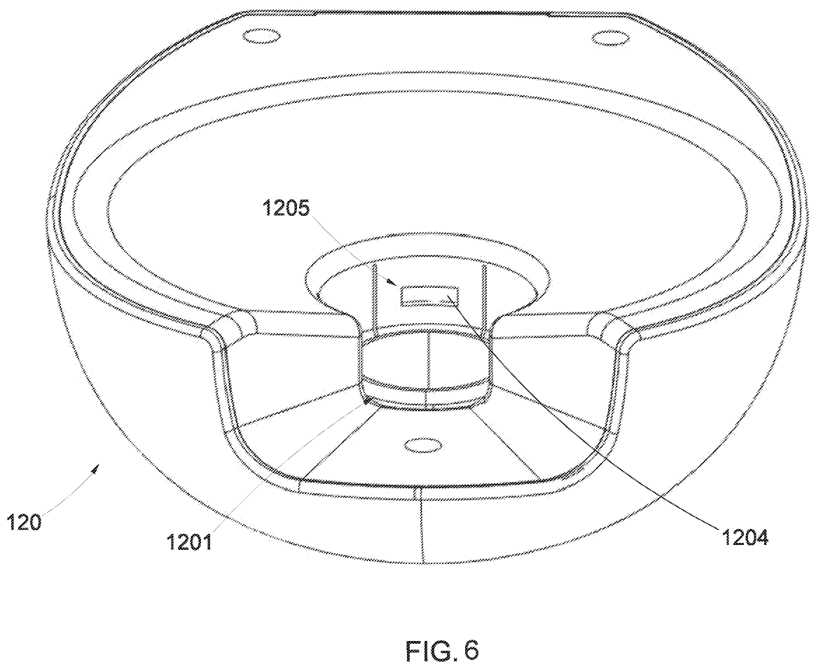

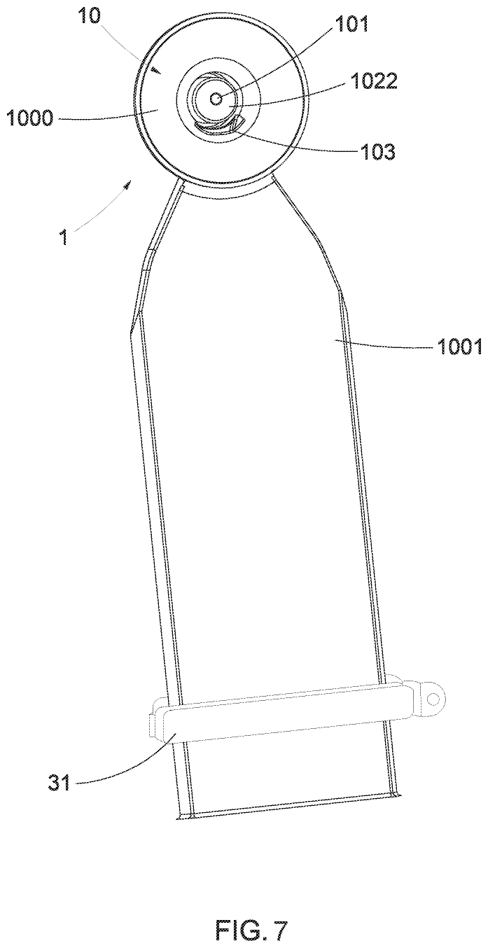

Reference is collectively to FIG. 1, FIG. 2, FIG. 3, FIG. 4, FIG. 5, FIG. 6 and FIG. 7, which are an exploded perspective view of the present invention, a perspective combined view of the present invention. The combined cross-sectional view of the present invention and the embodiment of the object to be sucked (breast, nipple), the top view of the main body of the present invention, the bottom view of the main body of the present invention, and the embodiment of the present invention in which the number collecting groove of the cylinder is provided with a sealing clip FIG. In a preferred embodiment, the silent negative pressure system of the present invention that does not need to be driven by a pump includes: a horn cover 1000 and a software 1.

One of the aforementioned horn covers 1000 is provided with a cylinder 1001 at its lower end, and the cylinder 1001 is provided with a collecting groove 2 (as shown in the first, second, third, fourth, fifth, sixth and seventh figures).

One of the aforementioned software 1 is set inside the speaker cover 1000, and the back end of the software 1. There is one or more coils 12 and a plug 1002, the center of the software 1 is provided with a cavity 11 for generating negative pressure, and the front end of the software 1 is provided with an opening 10 for the object to be sucked to fit and suck. After the software 1, the end extension is provided with a single or a plurality of elastic inner recesses located inside the cavity 11 and having a single or a plurality of negative pressure chambers and a single or a plurality of negative pressure chambers. The front surface of the elastic inner recess 102 forms a negative pressure chamber film and touch type. The prolactin film 1022, the end of the elastic inner recess 102 is provided with a magnet 101 corresponding to the coil 12; the inner section of the lower edge of the cavity 11 of the software 1 is provided with a flow channel 104 that communicates with the cavity 11 and communicates with the collecting tank 2 of the cylinder 1001; the middle section of the lower edge of the cavity 11 of the software is provided with a side channel 105 communicating with the cavity 11, and a blocking device is protruded from the upper end of the side channel 105 103; the lower end of the side flow channel 105 of the cavity 11 meets the lower end of the flow channel 104 and communicates with the collection tank 2 of the cylinder 1001 and is provided with a check valve 1031; and the coil 12 of the software 1 generates a magnetic field through the magnitude of the current and controls the magnitude of the magnetic force of the coil 12, controls the magnitude of the magnetic force through the magnitude of the current of the coil 12, and uses Ampere's law to drive the coil 12 with current to generate an attractive force for the magnet 101, so as to be pulled to the outside to generate negative pressure inside the cavity 11, and the current is stopped when it recovers, and the elastic inner recess 102 of the software 1 can be used to restore the stress and rebound or drive a reverse current to make the coil 12 generate a repulsive force and push it back. The magnet 101, in order to provide the collection of milk, in the present invention, when the collection groove 2 of the cylinder 1001 is used as a milk collection bag, it can be clamped by a sealing clip 31 at the lower end to collect milk as a bag body (such as FIG. 7 as shown in the figure), that is, the opening of the collection tank 2 is directly sealed to allow the milk to remain in the collection tank 2, and the sealing clip 31 is opened only when it needs to be poured out, and the check valve of the present invention 1031 can provide the outflow of the milk, even if it is turned upside down, the milk will not flow to the horn cover 1000; if the collecting tank 2 of the cylinder 1001 of the present invention does not have the sealing clip 31, it can be added to the cylinder. The collecting tank 2 of 1001 is provided with a milk collecting product 3, and the milk collecting product 3 can be a milk collecting bag or feeding bottle, but this does not limit the present invention; when the milk collecting bag is used, there is an opening 30 at one end, and the opening 30 provides the collecting groove 2 to fit the cylinder 1001, and is then clamped by a sealing clip 31 provided (such as the first, second, third As shown in the figure), the sealing clips 31 used in the collection tank 2 of the cylinder 1001 or in the milk collection bag of the milk collection product 3 are of different shapes; in this embodiment, the software 1 is made of soft material, which can conform to the skin and is not easy to leak even if it is reclined, but this does not limit the invention. In this embodiment, the blocking device 103 is a flange, which does not limit the invention. Invention; use the current to drive the coil 12 to generate a magnetic field, provide interaction with the magnet 101, deform the elastic inner recess 102 and generate a silent negative pressure in the cavity 11 (such as shown in FIGS. 1-7).

Furthermore, in the present invention, a host 120 is disposed behind the speaker cover 1000, and is disposed at the rear end of the speaker cover 1000. The host 120 has a circuit board 1200 and is provided with the slot embedded in the rear end of the software 1 at the front end. An insertion part 1201 of 1002 (as shown in the first, second, third, fourth, fifth, sixth and seventh figures); the side wall of the cylinder 1001 of the speaker cover 1000 is provided with a charging hole 1204 connected to the battery 12000, there are at least two indicator lights 1205 adjacent to the charging hole 1204. In this embodiment, the indicator lights 1205 are red or green lights, that is, red light is emitted when charging is in progress, and green light is emitted when fully charged (as shown in the sixth figure), the key 1202 includes a left key 12021, a middle key 12022, and a right key 12023 (as shown in the fifth figure); the host 120 is further provided with a display screen 1203, in this embodiment, the upper left section provides a display of boosting/pumping mode, the upper middle section provides a display of the remaining time (such as minutes: seconds), and the upper right section provides a display of battery power. The lower the number of black bars, the lower the battery level; the lower part of the display screen 1203 provides a display of the strength of suction, the smaller the number of black bodies, the weaker the suction; the operation of the left button 12021 of the key 1202 can produce the function of reducing the suction power by a short press alone, if the same as the middle button 12022 pressing the middle button 2022 for three seconds will activate the function of on or off, and if you press it for a short time, it will activate the function of mode switching; pressing the left button 12023 for a short time alone will activate the function of increasing suction. If it is pressed with the middle button 2022, it will have the function of increasing the time; use the current to drive the coil 12 to generate a magnetic field, provide interaction with the magnet 101, and deform the elastic inner recess 102 and the cavity 11 to generate silent negative pressure (as shown in the first, second, third and fifth and sixth pictures); when the user is expressing milk on the side, the milk can flow down the nipple (as shown in the fourth picture)); in the present invention, the host 120 can also be linked through an application program (APP) downloaded by a smart machine, and use various system functions.

When the present invention is used, it does not use a motor, but through the coil 12 and the magnet 101 disposed in the elastic inner recess 102 of the software 1, the coil 12 is used to attract the magnet 101 magnetically, so that the cavity can generate negative pressure. The body 11 is compressed and deformed, and drives the cavity 11 to generate negative pressure, so that the noise of the negative pressure generated by the motor drive of the habit is not generated. When the user is lying on the side to express milk, the milk can flow down along the nipple, and the cavity 11 of the software 1 is in close contact with the skin, and with the blocking device 103, the milk can flow into the collection tank 2 through the side channel 105.

### Advantages of the Present Invention

(1) Silence: It can pass through the elastic inner recess connected to the software 1 in a mute state 102 is deformed, so that the cavity 11 achieves a negative pressure effect.
(2) Cost: When using the motor to pump air in the home appliance technology, it is necessary to match the pressure relief valve to relieve the negative pressure at the same time. There are many components to be controlled in the circuit design, and the circuit board is larger, and the present invention only passes through the coil 12 and the magnet 101 provided in the elastic inner recess 102, the circuit is relatively simple and the cost is also low.
(3) Weight: the present invention omits the motor and the pressure relief valve, and only replaces it with the coil 12, while the circuit board 1200 is smaller, lighter, and more suitable for wearable devices.
(4) Volume: the circuit board 1200 of the present invention is small and does not require a motor and a pressure relief valve, so the host 120 is made more compact.
(5) No leakage: the software 1 has the cavity 11 and can conform to the skin, and it is not easy to leak even if it is reclining. When reclining and expressing milk, the milk may flow down along the nipple. When the software 1 is in close contact with the skin, with the blocking device 103, the milk can flow into the collection tank 2 along the way.

Compared with the prior art, the present invention is more novel and progressive, wherein, compared with the invention patent of the Republic of China No. 1675675, (Japanese Patent No. 6691591) “invisible milking device”, the structure of the conventional product includes a main unit, a The extrusion unit, milk collecting bag, stop valve unit and drive unit pump air through the motor in the main unit, so that the negative pressure chamber of the software generates negative pressure and then compresses and deforms, which drives the milk collecting chamber to generate negative pressure to suck out the milk. To generate noise, special sound insulation treatment is required to reduce the troubles of users as mentioned above; the present invention completely mutes the formation process of negative pressure through the mute method of magnetic force, so the present invention is more novel and progressive; Compared with the patent of the invention in mainland China: CN103182103B “Electric Breast Pump”, the structure of the habit is different from that of the present invention, and the negative pressure generation method of the present invention only needs to be placed in the back end of the software 1. The elastic inner recess 102 of the cavity 11 is embedded in the magnet 101, and the coil 12 is used to pass current to generate a magnetic field to attract or repel the magnet 101 and to adjust the negative pressure, which saves components and achieves stability, comfort and static. Therefore, the present invention is more novel and progressive; the present invention is compared with the invention patent of “A Negative Pressure Relief Device on a Breast Pump” No. CN109125822A. There are differences in the invention. The difference is that the habit is a kind of moderator derived from the negative pressure pressurization too fast, and the breakthrough technology of the present invention is mainly to replace the air pumping, only the coil 12 can make the device larger. The width is simplified, and the mute effect is achieved, so the present invention has novelty and progress; the present invention is compared with the invention patent of “Electronic Negative Pressure Pulsator for Milking” with the Chinese Mainland Publication No. CN107980642A, which is an electronic negative pressure pulsator for milking, which has a relatively complex structure and has the same advantages as the present invention. There are obvious structural differences. The breakthrough technology of the present invention is mainly to replace the air pumping. Only the coil 12 can greatly simplify the device and achieve a mute effect. Therefore, the present invention is novel and progressive; in addition, the present invention can also be used in electric nasal aspirator, because many children are very afraid of the sound of the motor, so parents often need to struggle with children when using the current nasal aspirator.

Therefore, the present invention utilizes the software 1 to extend from the rear end to have the magnet at the inner end of the elastic inner concave portion 102 of 101, the front end surface of the elastic inner concave portion 102 forms a negative pressure cavity film and a stroking pro-emulsifying film 1022, and the rear end of the software 1 is provided with the coil 12 acting with the magnet 101; so as to achieve, that the coil 12 is driven by current to generate a magnetic field, the elastic inner recess 102 can be deformed and the cavity 11 can generate a mute negative pressure; the convenience of use and mute effect are effectively improved, and the conventional motor and pressure relief valve are omitted, which will greatly expand the industry. The utilization is novel and progressive.

In sum, it can be seen that the present invention has indeed achieved the desired effect under the breakthrough of the previous technology, and it is not easy for those who are familiar with the art to think about it. Furthermore, the present invention has not been disclosed before the application. It is novel and progressive, and obviously meets the application requirements for an invention patent. It is advisable to file an invention application in accordance with the law. It is urged that the bureau to approve this invention patent application to encourage inventions.

The above-mentioned embodiments are only for illustrating the technical ideas and characteristics of the present invention, and their purpose is to enable those who are familiar with the art to understand the content of the present invention and implement them accordingly. The scope of the patent, that is, all equivalent changes or modifications made in accordance with the spirit disclosed in the present invention shall still be covered within the scope of the patent of the present invention.

1. A pumpless silent negative pressure system, characterized in that it comprises:
a horn cover, wherein the lower end of which is provided with a cylinder; a software arranged inside the horn cover, wherein the rear end of the software is provided with one or more coils,
wherein the center of the software is provided with a cavity for generating negative pressure, the front end of the software is provided with an opening for adhering to the object to be sucked, the rear end of the software is extended with a cavity inside the cavity and having a return stress rebound and a single or a single or a plurality of elastic inner concave parts of a plurality of negative pressure chambers, the front end surface of the elastic inner concave part forms a negative pressure chamber film and a stroking type promoting milk film, and the end of the elastic inner concave part is provided with a magnet corresponding to the coil;
wherein by using current to drive the coil to generate a magnetic field, interaction with the magnet is provided, the elastic inner recess is deformed and a silent negative pressure in the cavity is generated.

2. The pumpless silent negative pressure system as claim 1, wherein the inner section of the lower edge of the cavity of the software is provided with a flow channel that communicates with the cavity, and the cylinder of the horn cover is provided with a collection groove and communicated with the flow channel.

3. The pumpless silent negative pressure system as claim 2, wherein the middle section of the lower edge of the cavity of the software is provided with a side flow channel communicating with the cavity, and a blocking device is protruded from the upper end of the side flow channel.

4. The pumpless silent negative pressure system as claim 3, wherein the lower end of the side flow channel of the cavity meets the lower end of the flow channel and communicates with the collection tank and is provided with a check valve.

5. The pumpless silent negative pressure system as claim 1, wherein the coil of the software controls the magnetic force of the coil through the magnitude of the current.

6. The pumpless silent negative pressure system as claim 4, wherein the collecting tank of the cylinder is provided with a dairy collecting product.

7. The pumpless silent negative pressure system as claim 4, wherein a sealing clip is arranged at the lower end of the collecting tank of the cylinder.

8. The pumpless silent negative pressure system as claim 6, wherein the milk collection system is a milk collection bag or a feeding bottle.

9. The pumpless silent negative pressure system as claim 8, wherein this dairy collecting bag of described dairy collecting product is provided with an opening at its one end, and this opening is provided with a sealing clip.
